(19) United States
(12) Patent Application Publication
(10) Pub. No.: US 2019/0290139 A1
(43) Pub. Date: Sep. 26, 2019

(54) APPARATUS AND METHODS FOR DETERMINING PULSE WAVE VELOCITY USING MULTIPLE PRESSURE SENSORS

(71) Applicant: KONINKLIJKE PHILIPS N.V., EINDHOVEN (NL)

(72) Inventors: Charles Frederik SIO, Eindhoven (NL); Arjen VAN DER HORST, Tilburg (NL); Maarten Petrus Joseph KUENEN, Veldhoven (NL)

(21) Appl. No.: 16/301,485

(22) PCT Filed: May 19, 2017
(86) PCT No.: PCT/EP2017/062092

§ 371 Date: Nov. 14, 2018

(30) Foreign Application Priority Data
May 20, 2016 (EP) 16170646.0
Jun. 29, 2016 (EP) 16176873.4

(51) Int. Cl. A61B 5/0215; A61B 5/021; A61B 5/00

(52) U.S. Cl. CPC A61B 5/02158; A61B 5/6852; A61B 5/7264; A61B 5/02125

(57) ABSTRACT

Devices, systems and methods for pulse wave velocity determination in a renal artery are disclosed. An intravascular system may be included with two or more sensors disposed a certain distance apart on a flexible, elongate member. The sensors may be configured to receive pressure measurements associated with pulse waves moving through the renal artery, at different times. This difference in time and the distance between the 5 sensors may be used to calculate pulse wave velocity.

### TECHNICAL FIELD OF THE INVENTION

Embodiments of the present disclosure relate generally to the field of medical devices and, more particularly, to devices, systems, and methods for determining pulse wave velocity.

### BACKGROUND OF THE INVENTION

Hypertension and its associated conditions, chronic heart failure (CHF) and chronic renal failure (CRF), constitute a significant and growing global health concern. Current therapies for these conditions span the gamut covering non-pharmacological, pharmacological, surgical, and implanted device-based approaches. Despite the vast array of therapeutic options, the control of blood pressure and the efforts to prevent the progression of heart failure and chronic kidney disease remain unsatisfactory.

Blood pressure is controlled by a complex interaction of electrical, mechanical, and hormonal forces in the body. The main electrical component of blood pressure control is the sympathetic nervous system (SNS), a part of the body's autonomic nervous system, which operates without conscious control. The sympathetic nervous system connects the brain, the heart, the kidneys, and the peripheral blood vessels, each of which plays an important role in the regulation of the body's blood pressure. The brain plays primarily an electrical role, processing inputs and sending signals to the rest of the SNS. The heart plays a largely mechanical role, raising blood pressure by beating faster and harder, and lowering blood pressure by beating slower and less forcefully. The blood vessels also play a mechanical role, influencing blood pressure by either dilating (to lower blood pressure) or constricting (to raise blood pressure).

The importance of blood pressure in the kidneys is amplified because of the central electrical, mechanical, and hormonal role of the kidneys play. For example, the kidneys affect blood pressure by signaling the need for increased or lowered pressure through the SNS (electrical), by filtering blood and controlling the amount of fluid in the body (mechanical), and by releasing key hormones that influence the activities of the heart and blood vessels to maintain cardiovascular homeostasis (hormonal). The kidneys send and receive electrical signals from the SNS and thereby affect the other organs related to blood pressure control. They receive SNS signals primarily from the brain, which partially control the mechanical and hormonal functions of the kidneys. At the same time, the kidneys also send signals to the rest of the SNS, which may boost the level of sympathetic activation of all the other organs in the system, effectively amplifying electrical signals in the system and the corresponding blood pressure effects. From the mechanical perspective, the kidneys are responsible for controlling the amount of water and sodium in the blood, directly affecting the amount of fluid within the circulatory system. If the kidneys allow the body to retain too much fluid, the added fluid volume raises blood pressure. Lastly, the kidneys produce blood pressure regulating hormones including renin, an enzyme that activates a cascade of events through the renin-angiotensin-aldosterone system (RAAS). This cascade, which includes vasoconstriction, elevated heart rate, and fluid retention, may be triggered by sympathetic stimulation. The RAAS operates normally in non-hypertensive patients but may become overactive among hypertensive patients. The kidney also produces cytokines and other neurohormones in response to elevated sympathetic activation that may be toxic to other tissues, particularly the blood vessels, heart, and kidney. As such, overactive sympathetic stimulation of the kidneys may be responsible for much of the organ damage caused by chronic high blood pressure.

Thus, overactive sympathetic stimulation of the kidneys plays a significant role in the progression of hypertension, CHF, CRF, and other cardio-renal diseases. Heart failure and hypertensive conditions often result in abnormally high sympathetic activation of the kidneys, creating a vicious cycle of cardiovascular injury. An increase in renal sympathetic nerve activity leads to the decreased removal of water and sodium from the body, as well as increased secretion of renin, which leads to vasoconstriction of blood vessels supplying the kidneys. Vasoconstriction of the renal vasculature causes decreased renal blood flow, which causes the kidneys to send afferent SNS signals to the brain, triggering peripheral vasoconstriction and increasing a patient's hypertension. Reduction of sympathetic renal nerve activity, e.g., via renal neuromodulation or denervation of the renal nerve plexus, may reverse these processes.

Efforts to control the consequences of renal sympathetic activity have included the administration of medications such as centrally acting sympatholytic drugs, angiotensin converting enzyme inhibitors and receptor blockers (intended to block the RAAS), diuretics (intended to counter the renal sympathetic mediated retention of sodium and water), and beta-blockers (intended to reduce renin release). The current pharmacological strategies have significant limitations, including limited efficacy, compliance issues, and side effects. As noted, renal denervation is a treatment option for resistant hypertension. However, the efficacy of renal denervation may be very variable between patients. Recent studies indicate that the velocity of the pressure/flow pulse (pulse wave velocity or PWV) inside the main renal artery may be indicative of the outcome of renal denervation. The PWV in patients with resistant hypertension may be very high (e.g., more than 20 m/s), which may make it difficult to determine the PWV in the relatively short renal arteries (e.g., 5-8 cm in length).

While the existing treatments have been generally adequate for their intended purposes, they have not been entirely satisfactory in all respects. The devices, systems, and associated methods of the present disclosure overcome one or more of the shortcomings of the prior art.

US 2010/0113949 A1 discloses systems and methods for the measurement of the velocity of a pulse wave propagating within a body lumen using an intravascular elongate medical device. The elongate medical device can include a data collection device configured to collect pulse wave data at a location within the lumen. The data collection device is communicatively coupled with a velocity measurement system and configured to output the collected data to the velocity measurement system. The velocity measurement system is configured to calculate the velocity of the pulse wave based on the collection data.

WO 99/34724 A2 relates to devices and methods for determining tubular wall properties for improved clinical diagnosis and treatment. Advantageously, tubular wall characteristics are recorded that correspond to the distensibility and compliance of the tubular walls. More specifically, the document provides for quantitative determination of the pressure wave velocity (PWV) of blood vessels, thereby characterizing, (inter alia), the Young modulus, the distensibility, the compliance, and the reflection coefficient of aneurysms, lesioned and non-lesioned parts of blood vessels.

Y. C. Chiu et al., “Determination of pulse wave velocities”, American Heart Journal, Vol. 121, No. 5, May 1, 1991, report on a study that was designed to investigate the efficacy of four computerized algorithms in the determination of pulse wave velocities in invasive as well as in noninvasive pressure determinations.

US 2014/0012133 A1 discloses methods for determining effectiveness of the denervation treatment comprising tracking at least one of arterial wall movement, arterial blood flow rate, arterial blood flow velocity, blood pressure and arterial diameter at one or more selected locations in the renal artery over time, and assessing the effectiveness of said renal denervation treatment according to results obtained by tracking.

P. Lurz et al., “Aortic pulse wave velocity as a marker for arterial stiffness predicts outcome of renal sympathetic denervation and remains unaffected by the intervention”, European Heart Journal, Vol. 36, No. Suppl. 1, Aug. 1, 2015, assess the impact of baseline arterial stiffness as assessed by aortic pulse wave velocity (PWV) on blood pressure (BP) changes after renal sympathetic denervation (RSD) for resistant arterial hypertension as well as the potential of RSD to at least partially reverse increased aortic stiffness.

### SUMMARY OF THE INVENTION

The present disclosure describes calculation of a physiological quantity known as a pulse wave velocity (PWV). The PWV represents the pressure/flow wave of the blood through blood vessels of a patient as a result of the heart pumping. Recent studies indicate that the PWV within the renal artery, which is an artery that supplies blood to the kidney, is indicative of whether a therapy known as renal denervation will be successful in the patient. Renal denervation is often used to treat hypertension. As described in more detail herein, PWV can be calculated based on measurements of pressure within the vessel. Two or more sensors can be attached a known distance apart to a flexible, elongate member that is positioned within the vessel. The sensors measure pressure associated with blood pulses moving through the vessel, at different times. This difference in time and the distance between the sensors may be used to calculate pulse wave velocity. The calculated PWV for the patient can then be used to determine whether the patient is good candidate for treatment. For example, the PWV measurement result can be used to perform patient stratification for the renal denervation, before performing the treatment, by predicting the efficacy of renal denervation based on PWV.

In one embodiment, an apparatus for pulse wave velocity (PWV) determination in a vessel is provided. The apparatus includes an intravascular device configured to be positioned within the vessel, the intravascular device including: a flexible elongate member having a proximal portion and a distal portion; a first pressure sensor coupled to the distal portion of the flexible elongate member; and a second pressure sensor coupled to the distal portion of the flexible elongate member at a position spaced from the first pressure sensor by a first distance along a length of the flexible elongate member such that the first pressure sensor is configured to monitor pressure within the vessel at a first location and the second pressure sensor is configured to monitor pressure within the vessel at a second location spaced from the first location; and a processing system in communication with the intravascular device, the processing system configured to: receive first pressure data associated with the monitoring of the pressure at the first location within the vessel by the first pressure sensor; receive second pressure data associated with the monitoring of the pressure at the second location within the vessel by the second pressure sensor; and determine a pulse wave velocity of fluid within the vessel based on the received first and second pressure data. The vessel is a renal artery and the sampling frequency of the first and the second pressure sensor is 10 kHz or higher, more preferably, 20 kHz or higher, most preferably, 40 kHz or higher.

In one embodiment, a method of determining pulse wave velocity (PWV) in a vessel is identified. The method includes monitoring a pressure at a first location within the vessel with a first pressure sensor; monitoring a pressure at a second location within the vessel with a second pressure sensor, wherein the second location is spaced from the first location along a length of the vessel by a first distance; receiving first pressure data associated with the monitoring of the pressure at the first location within the vessel by the first pressure sensor; receiving second pressure data associated with the monitoring of the pressure at the second location within the vessel by the second pressure sensor; and determining a pulse wave velocity of fluid within the vessel based on the received first and second pressure data. The vessel is a renal artery and the sampling frequency of the first and the second pressure sensor is 10 kHz or higher, more preferably, 20 kHz or higher, most preferably, 40 kHz or higher.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagrammatic schematic view of an exemplary intravascular system.

FIG. 2 is a diagrammatic schematic view of another exemplary intravascular system.

FIG. 3 is a schematic diagram illustrating an intravascular device positioned within the renal anatomy.

FIG. 4 is a graph of pressure measurements associated with pulse waves travelling through a vessel.

FIG. 5A is a diagrammatic schematic view of an exemplary intravascular device within a vessel combined with a graph showing pressure curves within the vascular pathway at a first time.

FIG. 5B is a diagrammatic schematic view of the exemplary intravascular device of FIG. 5A combined with a graph showing pressure curves within the vessel at a second time.

FIG. 6 shows a comparison of two pressure measurements associated with pulse waves travelling through a vessel at two different locations within the vessel.

FIG. 7 is a diagrammatic schematic view of an exemplary intravascular device within a branched vessel combined with a graph showing pressure curves within the vessel.

FIG. 8 is a flowchart illustrating a method of calculating a pulse wave velocity.

FIG. 9 is a flowchart illustrating another method of calculating a pulse wave velocity.

FIG. 10 is a flowchart illustrating another method of calculating pulse wave velocity.

### DETAILED DESCRIPTION OF THE EMBODIMENTS

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The present disclosure relates generally to devices, systems, and methods for determining and measuring pulse wave velocity in a main renal artery prior to a renal denervation treatment. The velocity of the pressure/flow pulse (pulse wave velocity or PWV) inside the main renal artery may be predictive of the outcome of renal denervation. The PWV may be very high in resistive hypertension patients, which makes it very difficult to perform an accurate measurement of PWV in the relatively short renal arteries. One method to determine the PWV is by utilizing the “water hammer” equation to calculate the PWV from simultaneous pressure and flow velocity measurements inside the vessel during a reflection free period (e.g., early systole):

$$PWV = \frac{1}{ρ} \frac{dP}{dU} \qquad ( 1 )$$

Or, alternatively, in case this reflection free period cannot be used the following relation may be used that determines the PWV by summation over the whole cardiac cycle:

$$PWV = \frac{1}{ρ} \sqrt{\frac{\sum {dP}^{2}}{\sum {dU}^{2}}} \qquad ( 2 )$$

with ρ being the blood density and P and U the pressure and velocity, respectively. As noted, renal denervation is a treatment option for resistant hypertension. Selection of patients for whom this treatment will be beneficial have met limited success so far. However, recent studies indicate that the velocity of the pressure/flow pulse (pulse wave velocity or PWV) inside the main renal artery pre-treatment may be predictive of the outcome of renal denervation treatment. In some instances, embodiments of the present disclosure are configured to perform pulse wave velocity measurements of the renal artery for stratification of patients for renal artery denervation. Renal sympathetic activity may worsen symptoms of hypertension, heart failure, and/or chronic renal failure. In particular, hypertension has been linked to increased sympathetic nervous system activity stimulated through any of four mechanisms, namely (1) increased vascular resistance, (2) increased cardiac rate, stroke volume and output, (3) vascular muscle defects, and/or (4) sodium retention and renin release by the kidney. As to this fourth mechanism in particular, stimulation of the renal sympathetic nervous system may affect renal function and maintenance of homeostasis. For example, an increase in efferent renal sympathetic nerve activity may cause increased renal vascular resistance, renin release, and sodium retention, all of which exacerbate hypertension. As an example, thermal neuromodulation by either intravascular heating or cooling may decrease renal sympathetic activity by disabling the efferent and/or afferent sympathetic nerve fibers that surround the renal arteries and innervate the kidneys through renal denervation, which involves selectively disabling renal nerves within the sympathetic nervous system (SNS) to create at least a partial conduction block within the SNS.

Several forms of renal injury or stress may induce activation of the renal afferent signals (e.g., from the kidney to the brain or the other kidney). For example, renal ischemia, a reduction in stroke volume or renal blood flow, may trigger activation of renal afferent nerve activity. Increased renal afferent nerve activity results in increased systemic sympathetic activation and peripheral vasoconstriction (narrowing) of blood vessels. Increased vasoconstriction results in increased resistance of blood vessels, which results in hypertension. Increased renal efferent nerve activity (e.g., from the brain to the kidney) results in further increased afferent renal nerve activity and activation of the RAAS cascade, inducing increased secretion of renin, sodium retention, fluid retention, and reduced renal blood flow through vasoconstriction. The RAAS cascade also contributes to systemic vasoconstriction of blood vessels, thereby exacerbating hypertension. In addition, hypertension often leads to vasoconstriction and atherosclerotic narrowing of blood vessels supplying the kidneys, which causes renal hypoperfusion and triggers increased renal afferent nerve activity. In combination this cycle of factors results in fluid retention and increased workload on the heart, thus contributing to the further cardiovascular and cardio-renal deterioration of the patient.

Renal denervation, which affects both the electrical signals going into the kidneys (efferent sympathetic activity) and the electrical signals emanating from them (afferent sympathetic activity) may impact the mechanical and hormonal activities of the kidneys themselves, as well as the electrical activation of the rest of the SNS. Blocking efferent sympathetic activity to the kidney may alleviate hypertension and related cardiovascular diseases by reversing fluid and salt retention (augmenting natriuresis and diuresis), thereby lowering the fluid volume and mechanical load on the heart, and reducing inappropriate renin release, thereby interrupting the deleterious hormonal RAAS cascade.

By blocking afferent sympathetic activity from the kidney to the brain, renal denervation may lower the level of activation of the whole SNS. Thus, renal denervation may also decrease the electrical stimulation of other members of the sympathetic nervous system, such as the heart and blood vessels, thereby causing additional anti-hypertensive effects. In addition, blocking renal nerves may also have beneficial effects on organs damaged by chronic sympathetic over-activity, because it may lower the level of cytokines and hormones that may be harmful to the blood vessels, kidney, and heart.

Furthermore, because renal denervation reduces overactive SNS activity, it may be valuable in the treatment of several other medical conditions related to hypertension. These conditions, which are characterized by increased SNS activity, include left ventricular hypertrophy, chronic renal disease, chronic heart failure, insulin resistance (diabetes and metabolic syndrome), cardio-renal syndrome, osteoporosis, and sudden cardiac death. For example, other benefits of renal denervation may theoretically include: reduction of insulin resistance, reduction of central sleep apnea, improvements in perfusion to exercising muscle in heart failure, reduction of left ventricular hypertrophy, reduction of ventricular rates in patients with atrial fibrillation, abrogation of lethal arrhythmias, and slowing of the deterioration of renal function in chronic kidney disease. Moreover, chronic elevation of renal sympathetic tone in various disease states that exist with or without hypertension may play a role in the development of overt renal failure and end-stage renal disease. Because the reduction of afferent renal sympathetic signals contributes to the reduction of systemic sympathetic stimulation, renal denervation may also benefit other organs innervated by sympathetic nerves. Thus, renal denervation may also alleviate various medical conditions, even those not directly associated with hypertension.

The devices, systems, and methods described herein allow for the determination of PWV in the renal arteries. In particular, accurate determination of localized PWV values in the renal artery may be used to predict the effect of renal denervation in a patient and selection of patients for whom this procedure is likely beneficial.

The PWV may be predictive of the outcome of renal denervation in treating resistive hypertension. As described herein, the computing device can output the calculated PWV to a display. A clinician may make therapeutic and/or diagnostic decisions, taking the PWV into consideration, such as whether to recommend the patient for a renal denervation procedure. In some instances, the computer system can determine and output a therapy recommendation or a likelihood-of-success prediction to the display, based on the PWV and/or other patient data. That is, the computer system may utilize the PWV to identify which patients are more likely and/or less likely to benefit from renal denervation.

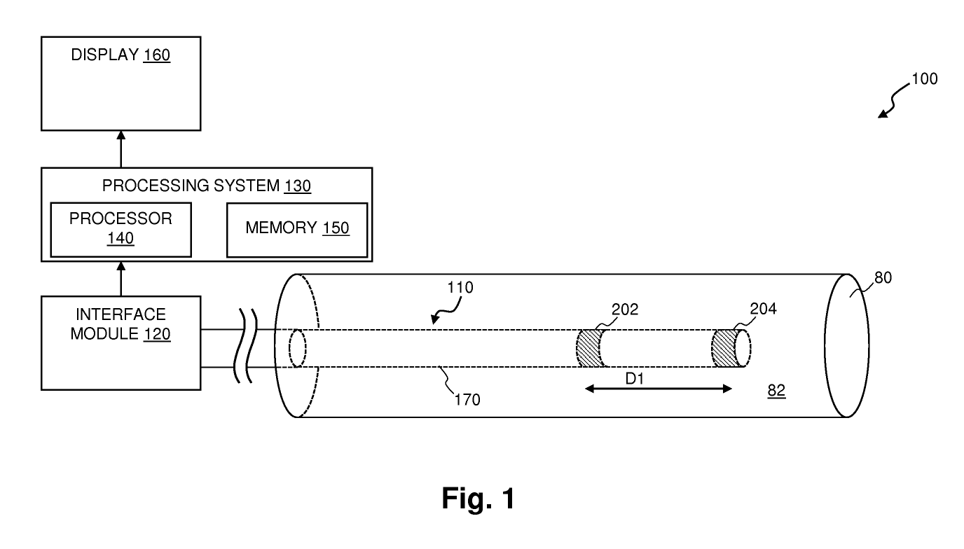

FIG. 1 is a diagrammatic schematic view of an exemplary intravascular system 100 according to some embodiments of the present disclosure. The intravascular system 100, which may be referred to as a stratification system, may be configured to perform pulse wave velocity (PWV) determination in a vessel 80 (e.g., artery, vein, etc.), for patient stratification for treatment purposes. For example, the PWV determination in the renal arteries may be utilized to determine whether a patient is suitable for renal artery denervation. The intravascular system 100 may include an intravascular device 110 that may be positioned within the vessel 80, an interface module 120, a processing system 130 having at least one processor 140 and at least one memory 150, and a display 160.

In some embodiments, the system 100 may be configured to perform pulse wave velocity (PWV) determination in a vessel 80 within a body portion. The intravascular system 100 may be referred to as a stratification system in that the PWV may be used for patient stratification for treatment purposes. For example, the PWV determination in the renal arteries may be utilized to determine whether a patient is suitable for renal artery denervation. Based on the PWV determination, the intravascular system 100 may be used to classify one or more patients into groups respectively associated with varying degrees of predicted therapeutic benefit of renal denervation. Any suitable number of groups or categories are contemplated. For example, the groups may include groups respectively for those patients with low, moderate, and/or high likelihood of therapeutic benefit from renal denervation, based on the PWV. Based on the stratification or classification, the system 100 can recommend the degree to which one or more patients are suitable candidates for renal denervation.

The vessel 80 may represent fluid-filled or surrounded structures, both natural and man-made. The vessel 80 may be within a body of a patient. The vessel 80 may be a blood vessel, as an artery or a vein of a patient's vascular system, including cardiac vasculature, peripheral vasculature, neural vasculature, renal vasculature, and/or or any other suitable lumen inside the body. For example, the intravascular device 110 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the heart, chambers or other parts of the heart, and/or other systems of the body. In addition to natural structures, the device intravascular 110 may be used to examine man-made structures such as, but without limitation, heart valves, stents, shunts, filters and other devices. Walls of the vessel 80 define a lumen 82 through which fluid flows within the vessel 80.

The vessel 80 may be located within a body portion. When the vessel 80 is the renal artery, the patient body portion may include the abdomen, lumbar region, and/or thoracic region. Generally, vessel 80 may be located within any portion of the patient body, including the head, neck, chest, abdomen, arms, groin, legs, etc.

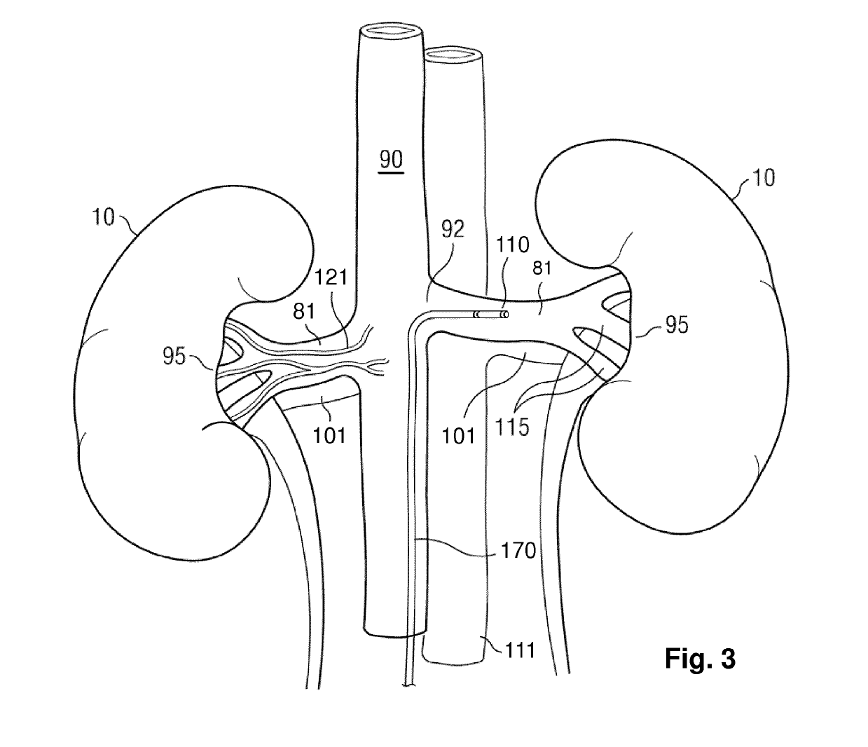

In some embodiments, the intravascular device 110 may include a flexible elongate member 170 such as a catheter, guide wire, or guide catheter, or other long, thin, long, flexible structure that may be inserted into a vessel 80 of a patient. In some embodiments, the vessel 80 is a renal artery 81 as shown in FIG. 3. While the illustrated embodiments of the intravascular device 110 of the present disclosure have a cylindrical profile with a circular cross-sectional profile that defines an outer diameter of the intravascular device 110, in other instances, all or a portion of the intravascular device may have other geometric cross-sectional profiles (e.g., oval, rectangular, square, elliptical, etc.) or non-geometric cross-sectional profiles. In some embodiments, the intravascular device 110 may or may not include a lumen extending along all or a portion of its length for receiving and/or guiding other instruments. If the intravascular device 110 includes a lumen, the lumen may be centered or offset with respect to the cross-sectional profile of the intravascular device 110.

The intravascular device 110, or the various components thereof, may be manufactured from a variety of materials, including, by way of non-limiting example, plastics, polytetrafluoroethylene (PTFE), polyether block amide (PEBAX), thermoplastic, polyimide, silicone, elastomer, metals, such as stainless steel, titanium, shape-memory alloys such as Nitinol, and/or other biologically compatible materials. In addition, the intravascular device may be manufactured in a variety of lengths, diameters, dimensions, and shapes, including a catheter, guide wire, a combination of catheter and guide wire, etc. For example, in some embodiments the flexible elongate member 170 may be manufactured to have length ranging from approximately 115 cm-155 cm. In one particular embodiment, the flexible elongate member 170 may be manufactured to have length of approximately 135 cm. In some embodiments, the flexible elongate member 170 may be manufactured to have an outer transverse dimension or diameter ranging from about 0.35 mm-2.67 mm (1 Fr-8 Fr). In one embodiment, the flexible elongate member 170 may be manufactured to have a transverse dimension of 2 mm (6 Fr) or less, thereby permitting the intravascular device 110 to be configured for insertion into the renal vasculature of a patient. These examples are provided for illustrative purposes only, and are not intended to be limiting. Generally, the intravascular device 110 is sized and shaped such that it may be moved inside the vasculature (or other internal lumen(s)) of a patient such that the pressure and cross-sectional area of a vessel 80 may be monitored from within the vessel 80.

In some embodiments, the intravascular device 110 includes a sensor 202 and a sensor 204 disposed along the length of the flexible elongate member 170. The sensors 202, 204 may be configured to collect data about conditions within the vessel 80, and in particular, monitor a pressure within the vessel 80. Furthermore, the sensors 202, 204 may periodically measure the pressure of fluid (e.g., blood) at the location of the sensors 202, 204 inside the vessel 80. In an example, the sensors 202, 204 are capacitive pressure sensors, or in particular, capacitive MEMS pressure sensors. In another example, sensors 202, 204 are piezo-resistive pressure sensors. In yet another example, sensors 202, 204 are optical pressure sensors. In some instances, the sensors 202, 204 include components similar or identical to those found in commercially available pressure monitoring elements such as the PrimeWire PRESTIGE® pressure guide wire, the PrimeWire® pressure guide wire, and the ComboWire® XT pressure and flow guide wire, each available from Volcano Corporation. In some embodiments, blood pressure measurements may be used to identify pulse waves passing through the vessel. The sensors 202, 204 may be disposed a first distance D1 apart. In some embodiments, the distance D1 is a fixed distance from 0.5 to 10 cm. In some embodiments, the distance D1 is within 0.5 to 2 cm. The distance D1 may be used in the calculation of Pulse Wave Velocity (PWV).

The sensors 202, 204 may be contained within the body of the intravascular device 110. The sensors 202, 204 may be disposed circumferentially around a distal portion of the intravascular device 110. In other embodiments, the sensors 202, 204 are disposed linearly along the intravascular device 110. The sensors 202, 204 may include one or more transducer elements. The sensor 202 and/or the sensor 204 may be movable along a length of the intravascular device 110 and/or fixed in a stationary position along the length of the intravascular device 110. The sensors 202, 204 may be part of a planar or otherwise suitably-shaped array of sensors of the intravascular device 110. In some embodiments, the outer diameter of the flexible elongate member 170 is equal to or larger than the outer diameter of the sensors 202, 204. In some embodiments, the outer diameter of the flexible elongate member 110 and sensors 202, 204 are equal to or less than about 1 mm, which may help to minimize the effect of the intravascular device 110 on pressure wave measurements within the vessel 80. In particular, since a renal artery generally has a diameter of approximately 5 mm, a 1 mm outer diameter of the intravascular device 110 may obstruct less than 4% of the vessel.

In some embodiments, one or both of the sensors 202, 204 may not be part of the intravascular device 110. For example, the sensor 204 may be coupled to a separate intravascular device or may be part of an external device. For example, the sensor 204 may be coupled to one of a guide wire or a catheter, and the sensor 202 may be coupled to the other of the guide wire or the catheter. In some instances, a first intravascular device having one of the sensors 202, 204 may be a guide wire, and the second intravascular device having the other of the sensors 202, 204 may be a catheter. The first and second intravascular devices can be positioned side by side within the vessel 80 in some embodiments. In some embodiments, a guide wire can at least partially extend through and be positioned within a lumen of the catheter such that the catheter and guide wire are coaxial. In case the two sensors are not disposed on the same device, the distance between the two sensors may be measured with methods for location of ultrasound transducers in the body by use of external ultrasound fields. Tracking the sensors of an interventional tool, e.g., the intravascular device 110, is disclosed in PCT Patent Application Publication No. WO2011138698A1 which is hereby incorporated in its entirety by reference.

The processing system 130 may be in communication with the intravascular device 110. For example, the processing system 130 may communicate with the intravascular device 110, including the sensor 202 and/or the sensor 204, through an interface module 120. The processor 140 may include any number of processors and may send commands and receive responses from the intravascular device 110. In some implementations, the processor 140 controls the monitoring of the pressure within the vessel 80 by the sensors 202, 204. In particular, the processor 140 may be configured to trigger the activation of the sensors 202, 204 to measure pressure at specific times. Data from the sensors 202, 204 may be received by a processor of the processing system 130. In other embodiments, the processor 140 is physically separated from the intravascular device 110 but in communication with the intravascular device 110 (e.g., via wireless communications). In some embodiments, the processor is configured to control the sensors 202, 204.

The processor 140 may include an integrated circuit with power, input, and output pins capable of performing logic functions such as commanding the sensors and receiving and processing data. The processor 140 may include any one or more of a microprocessor, a controller, a digital signal processor (DSP), an application specific integrated circuit (ASIC), a field-programmable gate array (FPGA), or equivalent discrete or integrated logic circuitry. In some examples, processor 140 may include multiple components, such as any combination of one or more microprocessors, one or more controllers, one or more DSPs, one or more ASICs, or one or more FPGAs, as well as other discrete or integrated logic circuitry. The functions attributed to processor 140 herein may be embodied as software, firmware, hardware or any combination thereof.

The processing system 130 may include one or more processors or programmable processor units running programmable code instructions for implementing the pulse wave velocity determination methods described herein, among other functions. The processing system 130 may be integrated within a computer and/or other types of processor-based devices. For example, the processing system 130 may be part of a console, tablet, laptop, handheld device, or other controller used to generate control signals to control or direct the operation of the intravascular device 110. In some embodiments, a user may program or direct the operation of the intravascular device 110 and/or control aspects of the display 160. In some embodiments, the processing system 130 may be in direct communication with the intravascular device 110 (e.g., without an interface module 120), including via wired and/or wireless communication techniques.

Moreover, in some embodiments, the interface module 120 and processing system 130 are collocated and/or part of the same system, unit, chassis, or module. Together the interface module 120 and processing system 130 assemble, process, and render the sensor data for display as an image on a display 160. For example, in various embodiments, the interface module 120 and/or processing system 130 generate control signals to configure the sensors 202, 204, generate signals to activate the sensors 202, 204, perform calculations of sensor data, perform amplification, filtering, and/or aggregating of sensor data, and format the sensor data as an image for display. The allocation of these tasks and others may be distributed in various ways between the interface module 120 and processing system 130. In particular, the processing system 130 may use the received pressure data to calculate a pulse wave velocity of the fluid (e.g., blood) inside the vessel 80. The interface module 120 can include circuitry configured to facilitate transmission of control signals from the processing system 130 to the intravascular device 110, as well as the transmission of pressure data from the intravascular device 110 to the processing system 130. In some embodiments, the interface module 120 can provide power to the sensors 202, 204. In some embodiments, the interface module can perform signal conditioning and/or pre-processing of the pressure data prior to transmission to the processing system 130.

The processing system 130 may be in communication with an electrocardiograph (ECG) console configured to obtain ECG data from electrodes positioned on the patient. ECG signals are representative of electrical activity of the heart and can be used to identify the patient's cardiac cycle and/or portions thereof. In some instances, the processing system 130 can utilize different formulas to calculate PWV based on whether the pressure data obtained by the intravascular device 110 is obtained over an entire cardiac cycle and/or a portion thereof. The ECG data can be used to identify the beginning and ending of the previous, current, and next cardiac cycle(s), the beginning and ending of systole, the beginning and ending of diastole, among other portions of the cardiac cycle. Generally, one or more identifiable features of the ECG signal (including without limitation, the start of a P-wave, the peak of a P-wave, the end of a P-wave, a PR interval, a PR segment, the beginning of a QRS complex, the start of an R-wave, the peak of an R-wave, the end of an R-wave, the end of a QRS complex (J-point), an ST segment, the start of a T-wave, the peak of a T-wave, and the end of a T-wave) can be utilized to select relevant portions of the cardiac cycle. The ECG console may include features similar or identical to those found in commercially available ECG elements such as the PageWriter cardiograph system available from Koninklijke Philips N.V.

Various peripheral devices may enable or improve input and output functionality of the processing system 130. Such peripheral devices may include, but are not necessarily limited to, standard input devices (such as a mouse, joystick, keyboard, etc.), standard output devices (such as a printer, speakers, a projector, graphical display screens, etc.), a CD-ROM drive, a flash drive, a network connection, and electrical connections between the processing system 130 and other components of the intravascular system 100. By way of non-limiting example, the processing system 130 may manipulate signals from the intravascular device 110 to generate an image on the display 160 representative of the acquired pressure data, imaging data, PWV calculations, and/or combinations thereof. Such peripheral devices may also be used for downloading software containing processor instructions to enable general operation of the intravascular device 110 and/or the processing system 130, and for downloading software implemented programs to perform operations to control, for example, the operation of any auxiliary devices coupled to the intravascular device 110. In some embodiments, the processing system 130 may include a plurality of processing units employed in a wide range of centralized or remotely distributed data processing schemes.

The memory 150 may be a semiconductor memory such as, for example, read-only memory, a random access memory, a FRAM, or a NAND flash memory. The memory 150 may interface with the processor 140 and associated processors such that the processor 140 may write to and read from the memory 150. For example, the processor 140 may be configured to receive data from the intravascular device 110 and/or the interface module 120 and write that data to the memory 150. In this manner, a series of data readings may be stored in the memory 150. The processor 140 may be capable of performing other basic memory functions, such as erasing or overwriting the memory 150, detecting when the memory 150 is full, and other common functions associated with managing semiconductor memory.

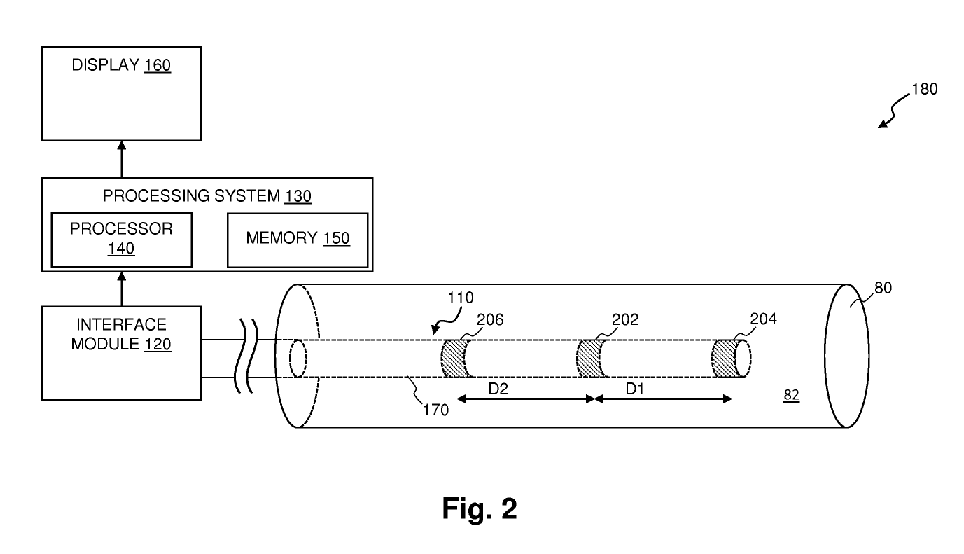

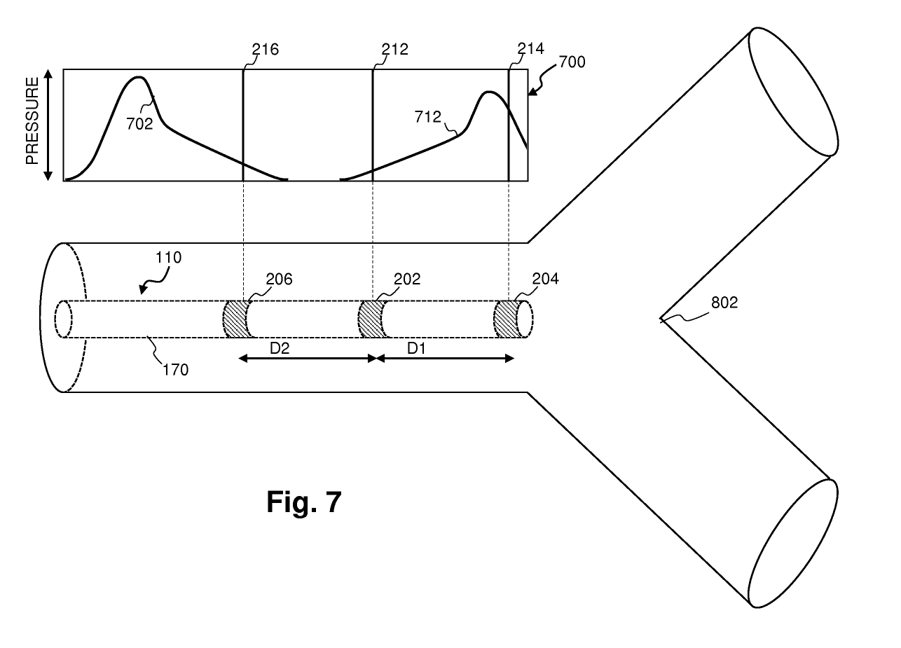

FIG. 2 is a diagrammatic schematic view of an exemplary intravascular system 180 according to some embodiments of the present disclosure. The intravascular system 180 may be similar to the intravascular system 100 of FIG. 1, with the addition of a third sensor 206. The intravascular systems 100, 180 as described herein may have four, five, six, or other numbers of sensors. The sensors may be placed in various orders and at different distances along the intravascular device 110. In some embodiments, the sensor 206 is disposed a distance D2 from the first sensor 202. The sensors 202, 204, 206 may also be placed in other arrangements and orders than that shown in FIG. 2. The sensor 206 may have a similar functionality to the sensors 202, 204 and may be used to measure the pressure within the vessel 80. In some embodiments, the sensor 206 may be used to determine the direction of travel of various pulse waves travelling through the vessel 80. The determination of the direction of travel may enhance the accuracy of PWV determinations by allowing the elimination of backwards-travelling pulse waves and associated data. The methods associated with direction of travel determination are discussed in more detail in relation to FIG. 7.

FIG. 3 illustrates the intravascular device 110 of FIG. 1 disposed within the human renal anatomy. The human renal anatomy includes kidneys 10 that are supplied with oxygenated blood by right and left renal arteries 81, which branch off an abdominal aorta 90 at the renal ostia 92 to enter the hilum 95 of the kidney 10. The abdominal aorta 90 connects the renal arteries 81 to the heart (not shown). Deoxygenated blood flows from the kidneys 10 to the heart via renal veins 101 and an inferior vena cava 111. Specifically, the flexible elongate member 170 of the intravascular device 110 is shown extending through the abdominal aorta and into the left renal artery 81. In alternate embodiments, intravascular device 110 may be sized and configured to travel through the inferior renal vessels 115 as well. Specifically, the intravascular device 110 is shown extending through the abdominal aorta and into the left renal artery 81. In alternate embodiments, the catheter may be sized and configured to travel through the inferior renal vessels 115 as well.

Left and right renal plexi or nerves 121 surround the left and right renal arteries 81, respectively. Anatomically, the renal nerve 121 forms one or more plexi within the adventitial tissue surrounding the renal artery 81. For the purpose of this disclosure, the renal nerve is defined as any individual nerve or plexus of nerves and ganglia that conducts a nerve signal to and/or from the kidney 10 and is anatomically located on the surface of the renal artery 81, parts of the abdominal aorta 90 where the renal artery 81 branches off the aorta 90, and/or on inferior branches of the renal artery 81. Nerve fibers contributing to the plexi arise from the celiac ganglion, the lowest splanchnic nerve, the corticorenal ganglion, and the aortic plexus. The renal nerves 121 extend in intimate association with the respective renal arteries into the substance of the respective kidneys 10. The nerves are distributed with branches of the renal artery to vessels of the kidney 10, the glomeruli, and the tubules. Each renal nerve 221 generally enters each respective kidney 10 in the area of the hilum 95 of the kidney, but may enter the kidney 10 in any location, including the location where the renal artery 81, or a branch of the renal artery 81, enters the kidney 10.

Proper renal function is essential to maintenance of cardiovascular homeostasis so as to avoid hypertensive conditions. Excretion of sodium is key to maintaining appropriate extracellular fluid volume and blood volume, and ultimately controlling the effects of these volumes on arterial pressure. Under steady-state conditions, arterial pressure rises to that pressure level which results in a balance between urinary output and water and sodium intake. If abnormal kidney function causes excessive renal sodium and water retention, as occurs with sympathetic overstimulation of the kidneys through the renal nerves 121, arterial pressure will increase to a level to maintain sodium output equal to intake. In hypertensive patients, the balance between sodium intake and output is achieved at the expense of an elevated arterial pressure in part as a result of the sympathetic stimulation of the kidneys through the renal nerves 121. Renal denervation may help alleviate the symptoms and sequelae of hypertension by blocking or suppressing the efferent and afferent sympathetic activity of the kidneys 10.

In some embodiments, the vessel 80 in FIG. 1 and FIG. 2 is a renal vessel consistent with the vessels 81 of FIG. 3 and the pulse wave velocity is determined in the renal artery. The processing system 130 may determine the pulse wave velocity (PWV) in the renal artery. The processing system 130 may determine a renal denervation therapy recommendation based on the pulse wave velocity in a renal artery. For example, patients that are more likely or less likely to benefit therapeutically from renal denervation may be selected based on the PWV. In that regard, based at least on the PWV of blood in the renal vessel, the processing system 130 can perform patient stratification for renal denervation.

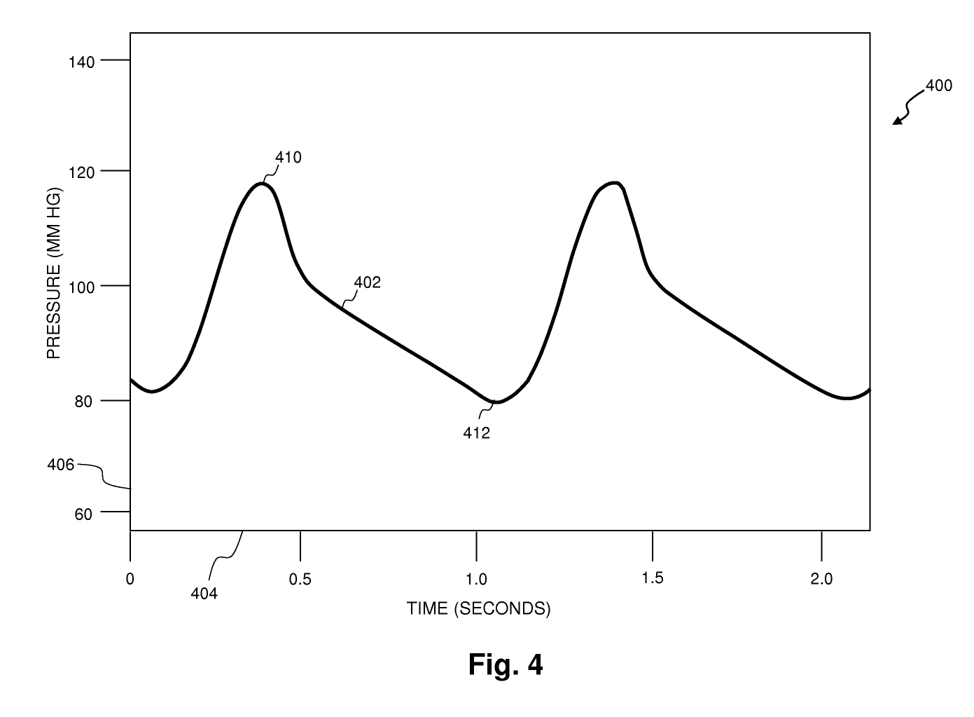

FIG. 4 is a graph 400 of pressure measurements associated with pulse waves travelling through a vessel. The graph 400 shows a pressure curve 402 of a fluid, e.g., blood, travelling through a vessel. The horizontal axis 404 may represent time and the vertical axis 406 may represent the fluid pressure in millimeters of mercury. For example, the graph 400 shows two complete pulses, each one taking about 1 second (corresponding to a heart rate of approximately 60 beats per minute). As an example, the pressure curve 402 may represent the pulse wave as a function of time at a specific point, e.g., the location of a sensor 202, 204, 206 inside the vessel 80. In some embodiments, pulse waves may be identified by certain aspects or characteristics of the pressure curve 402 include including peaks 410, troughs 412, notches (e.g., dicrotic notches), minimum values, maximum values, changes in values, and/or recognizable pattern(s). Additionally, the pulse waves may be identified by a foot-to-foot analysis or by dedicated analysis of the pulse arrival time from the pulse waveform, as described in Sold et al, Physiological Measurement, vol. 30, pp. 603-615, 2009, which is incorporated by reference herein in its entirety. Alternatively, more generic methods for time delay estimation may be adopted for the assessment of the time delay between the pressure waves, such as cross-correlation analysis, phase transform methods, maximum likelihood estimators, adaptive least mean squares filters, average squared difference functions, or the multiple signal classification (MUSIC) algorithm. In some embodiments, pressure sensors (such as the first, second, and third sensors shown in FIG. 2) may be configured to measure the presence and shape of pressure curves 402. This data may be used to determine the local PWV within a vessel 80. Optionally, the PWV value may then be used for stratification of patients with hypertension as eligible or ineligible for renal denervation.

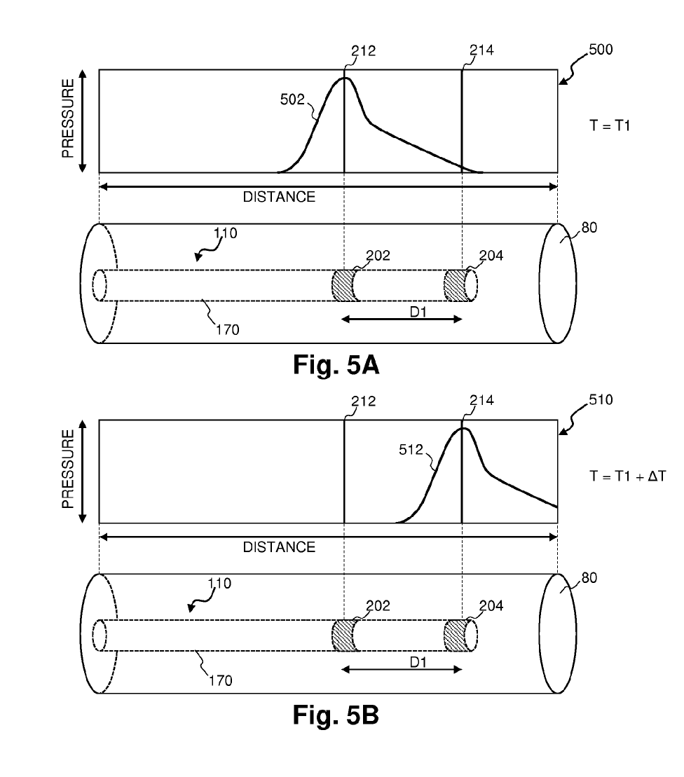

FIGS. 5A and 5B show perspective views of an exemplary intravascular device 110 within a vessel 80 combined with a graph showing a pressure curve within the vessel 80. The pressure curve may be associated with a pulse wave travelling through the vessel 80 as discussed in relation to FIG. 4. In the example of FIG. 5A, the graph 500 shows that the peak of the pressure curve 502 is aligned at point 212 with sensor 202 at time T1. FIG. 5B shows a graph 510 of a pressure curve 512 at a later time T2, where T2=T1+ΔT. The peak of the pressure curve 512 is aligned with the pressure sensor 204 at this point 214. Thus, in the time period ΔT the pulse wave has travelled the distance D1 between the sensor 202 and the sensor 204. By dividing this distance D1 by the time period ΔT, the PWV may be calculated. That is,

$$PWV = \frac{{D}_{1}}{Δ t} ,$$

where D₁ is the first distance and Δt is the amount of time between a pulse wave reaching the first location and the pulse wave reaching the second location. For example, the intravascular device 110 may include sensors 202, 204 disposed a distance D1 of 2 cm apart. The sensor 202 may detect a trough of a pulse wave at time T=0. The sensor 204 may detect the trough of the pulse wave at time T=1 ms, making a time period ΔT of 1 ms. The PWV may be calculated by dividing D1 by ΔT for a PWV of 20 m/s (0.02 m/0.001 s=20 m/s). While the peak pressure is shown in FIGS. 5A and 5B to determine ΔT, any identifiable feature or portion of the pulse wave may be utilized, including without limitation peaks, troughs, notches (e.g., dicrotic notches), minimum values (e.g. pressure, slope, etc.), maximum values (e.g. pressure, slope, etc.), changes in values, and/or recognizable pattern(s).

Due to the limited length of some vessels, such as the renal arteries 81, the sensors 202, 204 may be configured to measure pressures at high frequencies to provide better accuracy. For example, to achieve 90% accuracy of a PWV while using the data from the above example in the calculation of PWV, the intravascular system 100 must be able to distinguish between 20 m/s and 18 m/s. If the speed is 18 m/s, the time period ΔT between the pulse wave arriving at the sensors 202, 204 is (0.02 m)/(18 m/s)=1.11 ms. Therefore, in order to distinguish these PWV values, the intravascular system 100 must be able to distinguish between a time period ΔT of 1 ms and 1.11 ms, and thus distinguish in the order of about 0.1 ms.

Some existing pressure wire systems have a measurement frequency of 200 Hz (or one measurement every 5 ms) which is likely too low to achieve sufficient accuracy. However, the intravascular system 100 may be able to achieve sampling frequencies on the order of 50 kHz (one measurement every 0.02 ms), allowing a delay of 0.1 ms to be detected. In some embodiments, the intravascular system 100 may use a CMUT-on-ASIC pressure sensor such as that discussed in U.S. Pat. No. 8,617,088, which is incorporated herein in its entirety. Preferably, the sampling frequency of the first and the second pressure sensor 202, 204 is 10 kHz or higher, more preferably, 20 kHz or higher, most preferably, 40 kHz or higher. In some embodiments, the sampling frequency of the intravascular system 100 is between 10 and 80 kHz, between 20 and 70 kHz, or between 40 and 60 kHz. Other ranges of sampling frequencies are also possible.

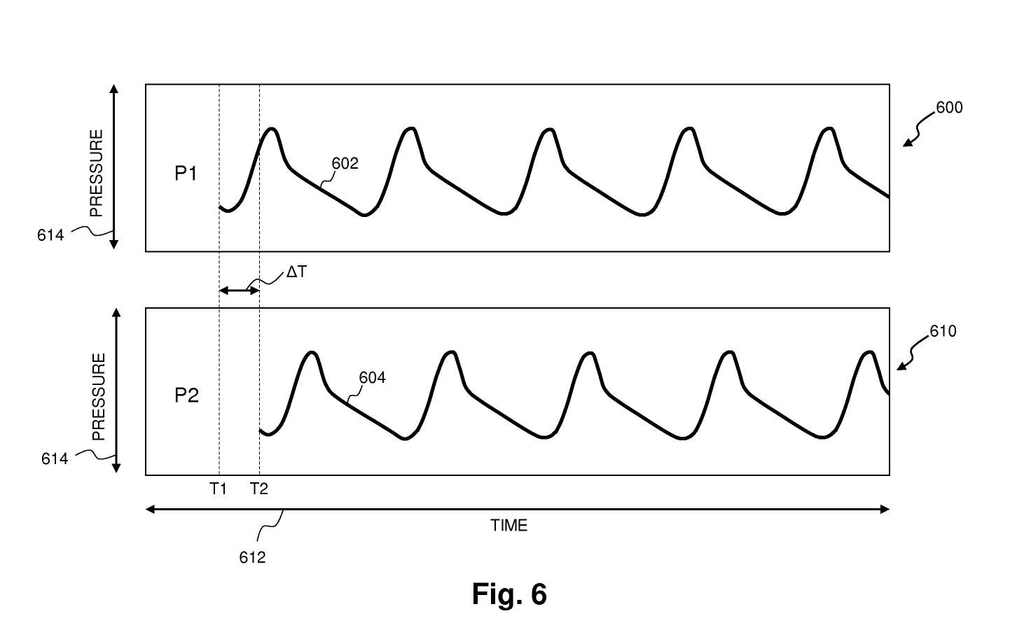

FIG. 6 shows a comparison of two pressure measurements associated with pulse waves travelling through a vessel at two different locations within the vessel. Graph 600 shows a pressure curve 602 of a fluid, e.g., blood, travelling through a vessel at a first location P1 within the vessel, while graph 610 shows a pressure curve 604 of the fluid at a second location P2 within the vessel. In some embodiments, the pressure curves 602, 604 are measured by pressure sensors such as the first and second sensors 202, 204.

In some instances, the second location P2 is distal or downstream of the fluid flow from the first location. The horizontal axes 612 of the graphs 600 and 610 may represent time and the vertical axes 614 may represent the fluid pressure in millimeters of mercury. As shown, the pressure curve 602 of graph 600 starts at time T1 and the pressure curve 604 of graph 610 starts at time T2, where ΔT=T2−T1 represents the time period it takes the pressure wave to travel from the first location associated with graph 600 to the second location associated with graph 610. In this manner, the graphs 600 and 610 of FIG. 6 illustrate a pulse wave traveling along a vessel where the pulse wave takes ΔT seconds to travel between first and second monitoring locations. This time period ΔT may be used to calculate the PWV of pulse waves in the vessel 80 as explained in reference to FIGS. 5A and 5B. It will be appreciated that the pressure curves 602, 604 may be compared by any number of aspects, such as peaks, troughs, slope measurements, curvature, areas with similar shapes, etc.

In some embodiments, the phase of the pressure curves 602, 604 may be identified by comparing the pressure differences between the measurements of the first and second sensors 202, 204 at a given time. For example, at the moment of arrival of a pressure curve 602, 604, the difference in pressures read by the first and second sensors 202, 204 may be close to zero. However, during the upslope of the pressure curve 602, 604, the pressure at first sensor 202 may be higher than the pressure at second sensor 204. Although the phase difference may be small (due to the short distance between the sensors), the pressure differences between the sensor readings may be higher because of the steep slope of the pressure curve 602, 604 during the upslope. As the pressure curve 602, 604 nears its peak over sensor 202, the difference in pressures will gradually decrease until it is a negative value. Near the end of the pressure curve 602, 604, the pressure is slowly dropping at sensor 202, meaning the pressure at first sensor 202 is lower than at sensor 204. The difference between sensors readings will give a small negative value and the phase difference between the two sensors is small.

In some embodiments, the activation of one or more of the first and second sensors 202, 204 is delayed such that the pressure curves 602, 604 measured by the first and second sensors 202, 204 have the same phase. The delay required to match the phase of the pressure curves 602, 604 is then used in the calculation of PWV. In some embodiments, the phase of the pressure curves 602, 604 may be determined by actuating the first and second sensors 202, 204 simultaneously and comparing the pressure readings from the sensors 202, 204. This method may include determining the delay by identifying when the difference between the pressure readings of the first and second sensors 202, 204 is zero. In some embodiments, the PWV is calculated from the slope of the pressure curve, the difference between the pressures measured at the two locations, and the distance D1 between the sensors. In some embodiments, the activation of the first and second sensors 202, 204 is controlled by one or more of the interface module 120 or processing system 130 (as shown in FIGS. 1 and 2), which may include delaying the activation of sensors for certain time periods.

In some embodiments, a third sensor 206 may also be included in the intravascular device 110, as shown in FIGS. 2 and 7. The sensor 206 may be selectively triggered so that the phase of the pressure curves 602, 604 is the same across all three sensors 202, 204, 206. This may provide for increased accuracy of PWV measurements because noise due to difference in the pressure curves 602, 604 may be minimized.

In some embodiments, the PWV may be determined by gating pressure curves 602, 604 through the use of an electrocardiogram (ECG) or one or more sensors disposed within the vessel 80. The ECG or additional sensors may be controlled by one or more of a separate system, an interface module 120, or a processing system 130, as shown in FIGS. 1 and 2. In particular, the velocity of pulse waves may be determined by analyzing the pressure curves 602, 604 synchronized by the ECG or additional sensors, such as an aortic pressure sensor. For example, as described herein, one or more feature of the ECG signal can be used to trigger data collection by the sensors. In some embodiments, pressure curves 602, 604 can be synchronized by performing mathematical analysis, such as a best fit analysis, to align the curves. The processing system 130, for example, can use the amount of the offset time required to bring the curves 602, 604 into alignment for synchronization.

In some instances, the interface module 120, as well as the processing system 130 can include a timer. By communicating to the interface module 120, the processing system 130 can synchronize the timer of the interface module 120 with the processor timer. Additionally, the interface module 120 can do the sampling of the signals received from sensors 202, 204 and can include a time stamp to the sampled data and then send the time-stamped sampled data to the processing system 130 such that the pressure data associated with the monitoring of the pressure within the vessel, received by processing system 130, is time-stamped and processing system 130 can synchronize the data based on the received time stamps.

Alternatively, instead of the interface module 120, the sensors 202, 204 can perform the sampling and send the sampled data to the processing system 130. The intravascular device 110 can include one or more timers for the sensors 202, 204. The processing system 130, by communicating to intravascular device 110, can synchronize data collection by the sensors 202, 204 with the processor timer. Thus, the data obtained by the sensors 202, 204 can include a time stamp. The interface module 120 can use the time stamps to synchronize the obtained data and then send the data to the processing system 130. In another example, the interface module 120 can send the time-stamped data obtained by sensors 202, 204 to the processing system 130. The processing system 130 can synchronize the data based on the received time stamps.

FIG. 7 is a perspective view of an exemplary intravascular device 110 within a branched vessel combined with a graph 700 showing pressure curves within the vessel 80. In some embodiments, pulse waves may be reflected within the vessel 80 for various reasons, including the presence of junctions or bifurcations in the vasculature. This reflection may cause pulse waves to travel in different directions through the vessel 80 which may interfere with the measurement of local PWV values. However, in some embodiments, the intravascular device 110 may include three or more sensors 202, 204, 206 which may allow for the identification and exclusion of backward-travelling pulse waves by monitoring the pressure at locations 212, 214, and 216, respectively. In particular, the third sensor 206 may be used to separate forward-travelling pulse waves (shown by pressure curve 702) from backward-travelling pulse waves (shown by pressure curve 712). In some embodiments, determining the directionality of the pulse waves may be accomplished by correlating pressure measurements from the three or more sensors 202, 204, 206 to identify the beginning and end of each pulse wave. The amplitude of the pulse waves may also be used in directionality determinations. For example, backward-travelling pulse waves such as that shown by pressure curve 702 may have a smaller amplitude than forward-travelling pulse waves such as that shown by pressure curve 712. In some embodiments, the separation of forward- and backward-travelling pulse waves may improve the accuracy of PWV calculations.

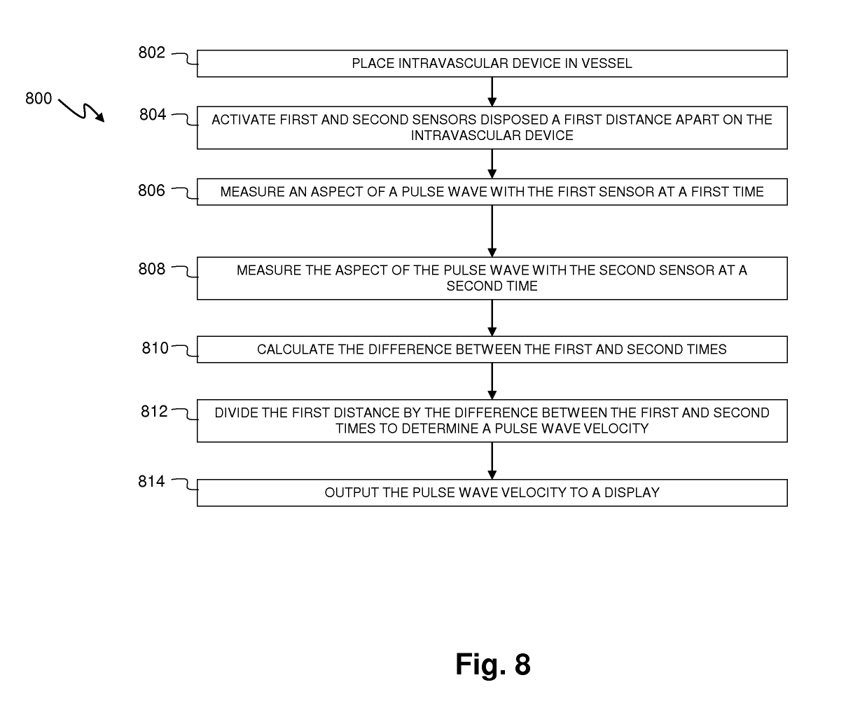

FIG. 8 is a flowchart illustrating a method 800 of calculating a pulse wave velocity (PWV). At step 802, the method 800 may include placing an intravascular device in a vessel. In some embodiments, the intravascular device is the intravascular device 110 shown in FIGS. 1, 2, 5A, 5B, and 7. The vessel may be a renal artery 81 as shown in FIG. 3.

At step 804, the method 800 may include activating first and second sensors disposed a first distance apart on the intravascular device. In some embodiments, the first and second sensors are pressure sensors. The first distance may be used in the calculation of the PWV. The first and second sensors may be disposed on a distal portion of a flexible, elongate device such as a catheter or guide wire.

At step 806, the method 800 may include measuring an aspect of a pulse wave with the first sensor at a first time. In some embodiments, this aspect may include a peak, trough, slope, foot, or other features of a pulse wave. The pulse wave may be identified by measuring the local pressure with the first and second sensors for a time period before the first time. This may allow for a complete view of the entire pulse wave and give an estimation of the time length and amplitude of the pulse waves in the vessel.

At step 808, the method 800 may include measuring the aspect of the pulse wave with the second sensor at a second time. At step 810, the method may include calculating the difference between the first and second times. This difference may be similar to the ΔT time period of FIGS. 5A, 5B, and 6. This calculation may be conducted by a controller in communication with the first and second sensors. In some embodiments, this aspect may include a peak, trough, notch, (e.g., dicrotic notch), minimum values, maximum values, curvature, changes in values, recognizable pattern(s), and/or other features of the pulse wave.

At step 812, the method 800 may include dividing the first distance by the difference between the first and second times to determine a PWV.

At step 814, the method 800 may optionally include outputting the PWV to a display. This display may be the display 160 shown in FIGS. 1 and 2. In some embodiments, the PWV may be used to evaluate the potential effect that renal denervation will have on a patient which may aid in selection of patients for whom renal denervation is likely beneficial.

In some embodiments, the method 800 optionally includes determining a therapy recommendation based on the PWV. In some instances, a clinician determines the therapy recommendation based on the computed PWV and/or other patient data. In some embodiments, the processing system evaluates the PWV and/or other patient data to determine the therapy recommendation. In such instances, the method 800 includes outputting a visual representation of the therapy recommendation. For example, the processing system can output display data associated with the graphical representation to a display device. This can be a textual indication, such as “Poor,” “Fair,” “Good,” and/or other suitable words may communicate the predicted benefit associated with therapy for the particular patient. In other instances, a numerical score, color coding, and/or other graphics representative of the therapy recommendation can be output to the display. The therapy can be renal denervation in some instances. The method 800 can additionally include classifying, based on the PWV, one or more patients into groups corresponding to respective degrees of predicted therapeutic benefit as a result of the renal denervation. The method 800 can also include the processing system outputting a graphical representation of the classifying step to the display device.

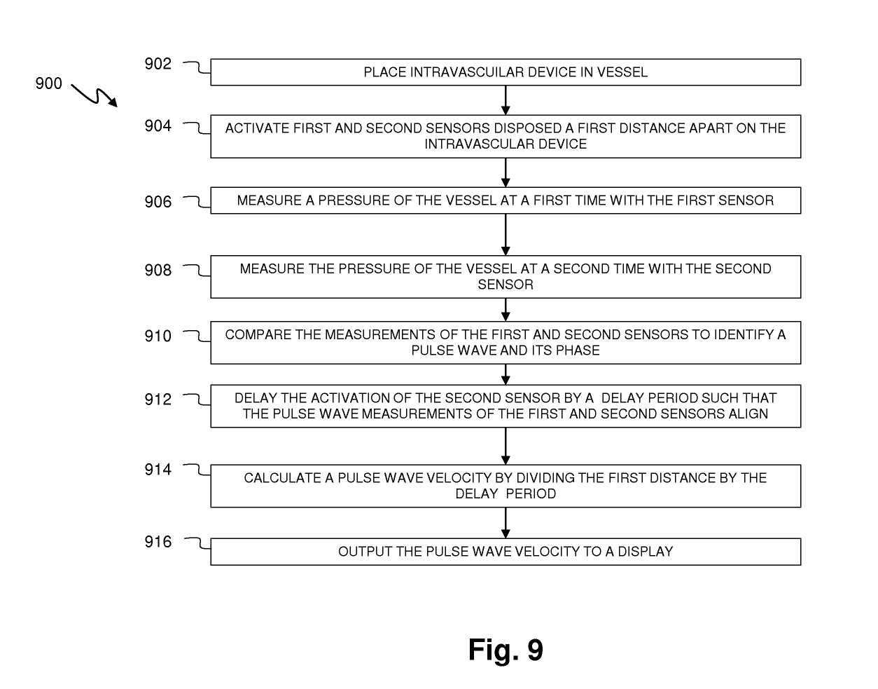

FIG. 9 is a flowchart illustrating a method 900 of calculating a PWV. At step 902, the method 900 may include placing an intravascular device in a vessel. In some embodiments, the intravascular device is the intravascular device 110 shown in FIGS. 1, 2, 5A, 5B, and 7. The vessel may be a renal artery 81.

At step 904, the method 900 may include activating first and second sensors disposed a first distance apart on the intravascular device. In some embodiments, the first and second sensors are pressure sensors. The first distance may be used in the calculation of the PWV. The first and second sensors may be disposed on a distal portion of a flexible, elongate device such as a catheter or guide wire.

At step 906, the method 900 may include measuring a pressure of the vessel with the first sensor at a first time. At step 908, the method 900 may include measuring the pressure of the vessel with the second sensor at a second time.

At step 910, the method 900 may include comparing the measurements of the first and second sensors to identify a pulse wave and its phase. In some embodiments, the pulse wave may be identified by analysis of the measurements and identification of aspects of a pulse wave such as peaks, troughs, slopes, or other feature. The pulse wave may be identified by measuring the local pressure for a time period before the first time. This may allow for a complete view of the entire pulse wave and give an estimation of the time length and amplitude of the pulse waves in the vessel.

At step 912, the method 900 may include delaying the activation of the second sensor by a delay period such that the pulse wave measurements of the first and second sensors align. In some embodiments, the activation of the first and second sensors is controlled by a controller.

At step 914, the method 900 may include calculating a PWV by dividing the first distance between the first and second sensors by the delay period. At step 916, the method 900 may optionally include outputting the PWV to a display. This display may be the display 160 shown in FIGS. 1 and 2. In some embodiments, the PWV may be used to evaluate the potential effect that renal denervation will have on a patient which may aid in selection of patients for whom renal denervation is likely beneficial.

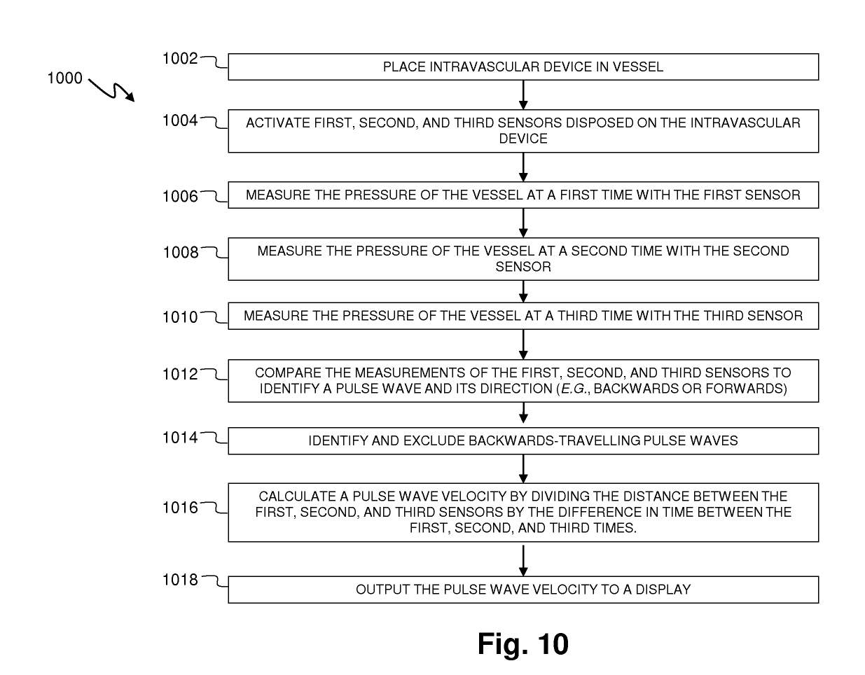

FIG. 10 is a flowchart illustrating a method 1000 of calculating a PWV. At step 1002, the method 1000 may include placing an intravascular device in a vessel. In some embodiments, the intravascular device is the intravascular device 110 shown in FIGS. 2 and 7. The vessel may be a renal artery 81.

At step 1004, the method 1000 may include activating first, second, and third sensors disposed on the intravascular device. In some embodiments, the first, second and third sensors are pressure sensors. The first distance may be used in the calculation of the PWV. The first, second, and third sensors may be disposed on a distal portion of a flexible, elongate device such as a catheter or guide wire. In some embodiments, each of the first, second, and third sensors may be disposed an equal distance from the other sensors.

At step 1006, the method 1000 may include measuring a pressure of the vessel with the first sensor at a first time. At step 1008, the method 1000 may include measuring the pressure of the vessel with the second sensor at a second time. At step 1010, the method 1000 may include measuring the pressure of the vessel with the third sensor at a third time.

At step 1012, the method 1000 may include comparing the measurements of the first, second, and third sensors to identify a pulse wave and its direction of travel (e.g., backwards or forwards). At step 1014, the method 1000 may include identifying and excluding backwards-travelling pulse waves from the data collected.

At step 1016, the method 1000 may include calculating a PWV by dividing the distance between the first, second, and/or third sensors by the difference in time between the first, second, and/or third times. In an embodiment, the three sensors can be used for both a better determination of the pulse wave velocity as for distinguishing of the forward and backward waves. At step 1018, the method 1000 may optionally include outputting the PWV to a display. This display may be the display 160 shown in FIGS. 1 and 2. In some embodiments, the PWV may be used to evaluate the potential effect that renal denervation will have on a patient which may aid in selection of patients for whom renal denervation is likely beneficial.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. An apparatus for pulse wave velocity (PWV) determination in a vessel, the apparatus comprising:
an intravascular device configured to be positioned within the vessel, the intravascular device including:
a flexible elongate member having a proximal portion and a distal portion;
a first pressure sensor coupled to the distal portion of the flexible elongate member; and
a second pressure sensor coupled to the distal portion of the flexible elongate member at a position spaced from the first pressure sensor by a first distance along a length of the flexible elongate member such that the first pressure sensor is configured to monitor pressure within the vessel at a first location and the second pressure sensor is configured to monitor pressure within the vessel at a second location spaced from the first location; and
a processing system in communication with the intravascular device, the processing system configured to:
receive first pressure data associated with the monitoring of the pressure at the first location within the vessel by the first pressure sensor;
receive second pressure data associated with the monitoring of the pressure at the second location within the vessel by the second pressure sensor; and
determine a pulse wave velocity of fluid within the vessel based on the received first and second pressure data,
wherein the vessel is a renal artery and the sampling frequency of the first and the second pressure sensor is 10 kHz or higher, more preferably, 20 kHz or higher, most preferably, 40 kHz or higher.

2. The apparatus of claim 1, wherein the first pressure sensor and/or the second pressure sensor is a CMUT-on-ASIC pressure sensor.

3. The apparatus of claim 1, wherein the pulse wave velocity is determined as
$$\frac{{D}_{1}}{Δ t} ,$$
where D₁ is the first distance and Δt is an amount of time between a pulse wave reaching the first location and the pulse wave reaching the second location.

4. The apparatus of claim 3, wherein an identifiable feature of the first and second pressure data is utilized to determine the amount of time between the pulse wave reaching the first and second locations.

5. The apparatus of claim 4, wherein the identifiable feature is at least one of:
a maximum pressure, a minimum pressure, or a slope.

6. The apparatus of claim 1, wherein the processing system is further configured to:
determine a renal denervation therapy recommendation based on the determined pulse wave velocity.

7. The apparatus of claim 1, wherein the processing system is further configured to:
classify a patient based on a predicted therapeutic benefit of renal denervation using the pulse wave velocity.

8. The apparatus of claim 1, wherein the intravascular device further includes:
a third pressure sensor coupled to the distal portion of the flexible elongate member at a position spaced from the first and second pressure sensors such that the third pressure sensor is configured to monitor pressure within the vessel at a third location spaced from the first and second locations.

9. A method of determining pulse wave velocity (PWV) in a vessel, comprising:
monitoring a pressure at a first location within the vessel with a first pressure sensor;
monitoring a pressure at a second location within the vessel with a second pressure sensor, wherein the second location is spaced from the first location along a length of the vessel by a first distance;
receiving first pressure data associated with the monitoring of the pressure at the first location within the vessel by the first pressure sensor;
receiving second pressure data associated with the monitoring of the pressure at the second location within the vessel by the second pressure sensor; and
determining a pulse wave velocity of fluid within the vessel based on the received first and second pressure data,
wherein the vessel is a renal artery and the sampling frequency of the first and the second pressure sensor is 10 kHz or higher, more preferably, 20 kHz or higher, most preferably, 40 kHz or higher.

10. The method of claim 9, wherein the first pressure sensor and/or the second pressure sensor is a CMUT-on-ASIC pressure sensor.

11. The method of claim 9, wherein the pulse wave velocity is determined as
$$\frac{{D}_{1}}{Δ t} ,$$
where D₁ is the first distance and Δt is an amount of time between a pulse wave reaching the first location and the pulse wave reaching the second location.

12. The method of claim 11, wherein an identifiable feature of the first and second pressure data is utilized to determine the amount of time between the pulse wave reaching the first and second locations.

13. The method of claim 12, wherein the identifiable feature is at least one of: a maximum pressure, a minimum pressure, or a slope.

14. The method of claim 9, further comprising synchronizing activation of the first and second pressure sensors.

15. The method of claim 14, wherein the synchronizing is based at least one of:
an ECG signal, an aortic pressure sensor reading, or a time difference between a pulse wave reaching the first location and the pulse wave reaching the second location.

16. The method of claim 9, the method further comprising:
determining a renal denervation therapy recommendation based on the determined pulse wave velocity.

17. The method of claim 9, the method further comprising:
classifying a patient based on a predicted therapeutic benefit of renal denervation using the pulse wave velocity.
